(19) United States
(12) Patent Application Publication
(10) Pub. No.: US 2010/0112707 A1
(43) Pub. Date: May 6, 2010

(54) Biosensor

(76) Inventors: Markku Känsäkoski, Oulu (FI); Liisa Kivimäki, Oulu (FI); Arto Maaninen, Oulu (FI); Marika Kurkinen, Oulu (FI); Kristiina Takkinen, Oulu (FI); Timo Pulli, Oulu (FI); Sanna Uusitalo, Oulu (FI); Sanna Aikio, Oulu (FI); Harri Siitari, Oulu (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 12/307,164

(22) PCT Filed: Jul. 4, 2007
(86) PCT No.: PCT/FI07/50412

§ 371 Date: Sep. 17, 2009

(30) Foreign Application Priority Data
Jul. 5, 2006 (FI) 20065478

(51) Int. Cl. G01N 33/68; B32B 38/04; G01N 33/48; G01N 21/00; G01N 25/00

(52) U.S. Cl. 436/86; 156/268; 422/68.1; 422/82.05; 422/82.12; 436/164; 436/147

(57) ABSTRACT

The invention relates to a biosensor solution in which the biosensor comprises at least one sol-gel response region pattern doped with a biological signature molecule and printed on the biosensor, and at least one micro-channel for transporting material to said at least one sol-gel response region pattern.

### FIELD

The invention relates to a method for analysing material, to a biosensor, and to a method for manufacturing a biosensor.

### BACKGROUND

In material analysis, an analyte in a material can be identified by means of a reagent containing a signature molecule or structure. From the interaction between the analyte and reagent, it is possible to determine the presence and possible amount of the analyte in the material.

Reference publication WO 01/81921 discloses a biosensor that comprises a polymer film and an analyte-specific binder layer ink-jet printed in a pattern on the polymer film. The interaction between the analyte and binder can be detected optically.

Reference publication WO2004/039487 discloses a multi-component protein microarray that has several spots of at least two different protein molecules bound in a biocompatible material and is used to study reactions.

Drawbacks in the prior-art solution include material supply that requires expensive liquid processing automatons or precise pipetting. Other problems are related to the patterning of the biocompatible material with a slow pinspotting method.

### BRIEF DESCRIPTION

It is an object of the invention to implement a method for analysing material, a biosensor, and a method for manufacturing a biosensor so as to provide a biosensor that is easy to manufacture and requires little processing of material prior to the analysis.

A first aspect of the invention comprises a biosensor for analysing material, which comprises at least one sol-gel response region pattern doped with a biological signature molecule and printed on the biosensor and at least one micro-channel for transporting the material to said at least one sol-gel response region pattern.

A second aspect of the invention comprises a method for analysing material, which comprises supplying the material to a micro-channel and transporting the material in the micro-channel to a sol-gel response region pattern doped with at least one biological signature molecule and printed on the biosensor.

A third aspect of the invention comprises a method for manufacturing a biosensor that comprises forming in the biosensor at least one micro-channel for transporting material and printing on the biosensor a sol-gel response region pattern doped with at least one biological signature molecule and connected to said at least one micro-channel.

Preferred embodiments of the invention are disclosed in the dependent claims.

The invention is based on the idea that the biosensor comprises a printed sol-gel response region pattern on which a biological signature molecule is doped before the printing. The sample is transported to the sol-gel response region pattern along a micro-channel.

The biosensor, analysis method and manufacturing method of the invention provide several advantages. The use of a micro-channel makes it possible to easily transport a sample to sol-gel response region patterns and to freely position sol-gel response region patterns on the biosensor, whereby it is easy to measure the sol-gel response region patterns. Printing the sol-gel response region pattern on a biosensor by using printing methods, for instance, makes the mass-production of biosensors possible.

### LIST OF FIGURES

The invention will now be described in greater detail by means of preferred embodiments and with reference to the attached drawings, in which

FIG. 1 shows a first example of the structure of the biosensor;

FIG. 2 shows an example of the structure of the micro-channel in one embodiment;

FIG. 3 shows a second example of the structure of the biosensor;

FIG. 4 shows a third example of the structure of the biosensor;

FIG. 5 shows a first example of an embodiment of the invention;

FIG. 6 shows a second example of an embodiment of the invention;

FIG. 7 shows a third example of an embodiment of the invention.

### DESCRIPTION OF THE EMBODIMENTS

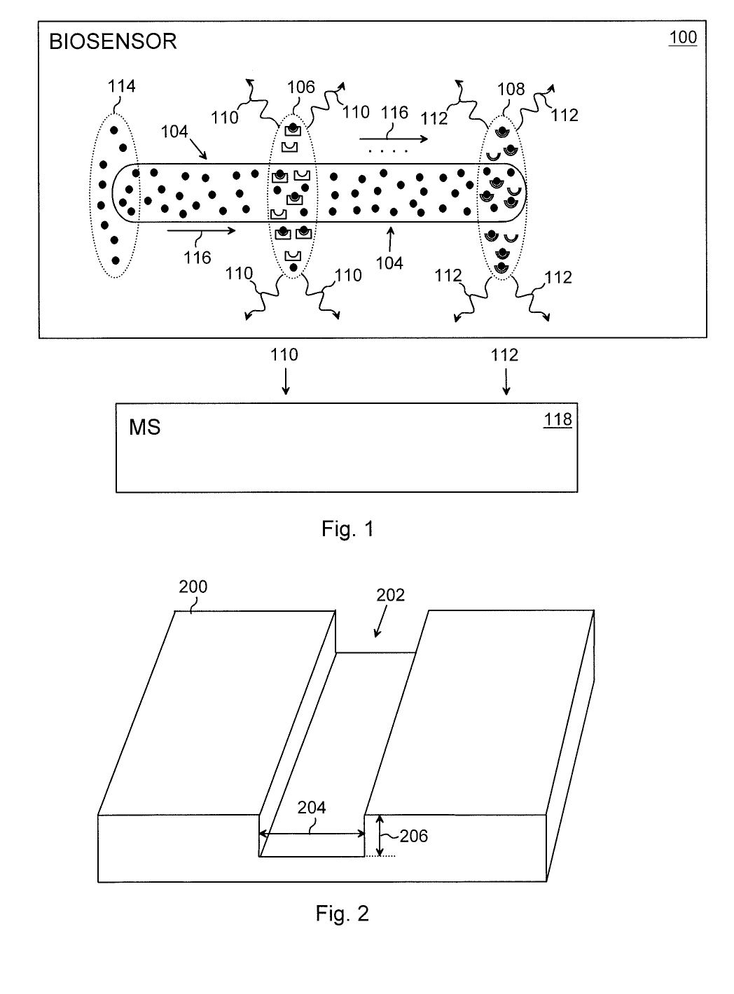

With reference to FIG. 1, an example of a biosensor 100 is examined, the biosensor comprising sol-gel response region patterns 106, 108 doped with a biological signature molecule. The biosensor 100 also comprises a micro-channel 104 that transports analyte components (marked with black circles) of the material to the sol-gel response region patterns 106, 108 in flow direction 116. FIG. 1 also shows a supply region 114 that receives material and supplies it to the micro-channel 104. The biosensor 100 can comprise one or more sol-gel response region patterns 106, 108. Each sol-gel response region pattern 106, 108 may have a pattern-specific signature molecule. Each sol-gel response region pattern 106, 108 may then be analyte-specific. In this context, the biological signature molecule is referred to a signature molecule in short.

The biosensor 100 can comprise a laminated structure, in which the micro-channel 104, sol-gel response patterns 106, 108 and/or supply region 114 are between the substrate and lamination cover part of the biosensor 100. The lamination cover part is not shown in FIG. 1.

FIG. 1 also shows a measuring device (MS) 118 for measuring the response 110, 112 of the signature molecule. The measuring device 118 measures the response from the sol-gel response region pattern 106, 108 and converts it into digital information. The measuring device 118 may be a portable device, such as a mobile phone or part thereof.

In solid form, the sol-gel medium is typically a ceramic-type material whose transition from liquid to solid form is achieved at temperatures at which signature molecules retain their activity.

In one embodiment, the sol-gel medium is made up of one or all of the following basic materials: alkoxy silane, such as glycidoxy propyl trimethoxy silane (GPTS), tetraethoxy silane (TEOS), tetramethoxy silane (TMOS), propyl trimethoxy silane (PTMS), methyl trimethoxy silane (MTMOS), ethyl acetoacetate (EtAcAc), titan isopropoxide (Ti(OPri)4), sodium silicate, chlorosilane, and catalysts, such as boehmite (AIO(OH)), and additives, such as polyvinyl alcohol (PVA), polyethylene glycol (PEG), and Tween 20.

Important properties of the sol-gel medium include compatibility with the signature molecule. The hardening temperature and pH of the sol-gel medium is then selected in such a manner, for instance, that the signature molecule retains its activity. In addition, the sol-gel medium is preferably porous so that the analyte and signature molecule can bind. Also, the sol-gel material preferably shrinks in moderation when gelating, endures the signature materials and does not dissolve or crumble.

The signature molecule is a reagent, such as cell, protein, peptide, enzyme, aptamer, MIP (molecular imprinted polymer), single-stranded DNA or RNA sequence. The signature molecule can be a natural or synthetic signature molecule whose reagent property is based on a natural reagent mechanism.

In one embodiment, the signature molecule is an antibody or antibody fragment, or an antibody or antibody fragment produced by gene technology processes (recombinant antibody). An advantage of antibodies is that they are identifiable, and they are used as commercial reagents in the ELISA (enzyme-linked immunosorbent assay) process, for instance.

The biosensor 100 is manufactured by doping signature molecules in a liquid sol-gel medium, and the sol-gel response region patterns 106, 108 are printed on the surface of the biosensor 100 substrate while the sol-gel medium is in liquid form. The signature molecules can distribute homogenously into the sol-gel medium. If it is necessary to form in the biosensor 100 several sol-gel response region patterns 106, 108 each having a different signature molecule, each signature molecule is mixed with separate sol-gel doses. The doses are printed on different regions of the biosensor 100, thus forming sol-gel response region patterns 106, 108 each of which has a specific signature molecule. In FIG. 1, the different signature molecules are marked with cup-like symbols. One sol-gel response region pattern 106, 108 may have one or more different doped signature molecules. If the signature molecules dope homogenously into the liquid sol-gel medium, it is possible to obtain a homogenous signature molecule distribution in a sol-gel response region pattern 106, 108. The sol-gel medium also protects the signature molecules in its inner layers from effects of the environment, for instance from heat and acidity. The substrate 200 can be covered with a cover, whereby a lamination structure is formed in the biosensor.

In printing the sol-gel response region patterns 106, 108, it is for instance possible to use an ink transfer method, such as gravure printing, inkjet printing and/or drop dosing. After printing, the sol-gel response region patterns 106, 108 are hardened into solid form by means of heat treatment or radiation, for instance. Relative to printing, sol-gel response region patterns 106, 108 doped with different signature molecules are an analogue concept for the colours used in ink printing. As the solvent in the sol-gel medium evaporates, pores with signature molecules on their inner surfaces are typically formed in the solid sol-gel medium. The effective surface area of the sol-gel response region patterns 106, 108 then becomes large, whereby a high sensitivity is achieved in material analysis.

The signature molecule doped in the sol-gel response region pattern 106, 108 has a measurable response 110, 112 with a previously known analyte. The material may or may not contain the analyte.

The measurable response 110, 112 can be an optical radiation emission from the sol-gel response region pattern 106, 108, a change in the optical reflection coefficient in the response region pattern 106, 108, a change in the permittivity in the response region pattern 106, 108, a thermal change in the response region pattern 106, 108, and/or a mechanical change in the response region pattern 106, 108. The response 110, 112 is based on the interaction between the material analyte and signature molecule.

The interaction can be based on bonding, for instance. The bonding mechanism can be a competitive or non-competitive immunoassay, for instance.

An optical radiation emission can be based on fluorescence, in which the analyte is marked with a fluorescent molecule. The analyte bonded with the signature molecule then emits fluorescent radiation in the sol-gel response region pattern 106, 108.

In one embodiment, the radiation emission is based on the FRET (fluorescence/Förster resonance energy transfer) mechanism. The analyte is then labelled with a molecule that fluoresces the analyte, and the signature molecule is labelled with a molecule that fluoresces the signature molecule. The emission bands of the molecule that fluoresces the analyte and the molecule that fluoresces the signature molecule overlap at least partly, whereby the fluorescent component having the shorter emission wavelength pumps energy into the fluorescent component having the longer emission wavelength and produces radiation emission from the fluorescent component having the longer emission wavelength. The radiation emission indicates the interaction between the analyte and signature molecule.

A change in the optical reflection coefficient in the response region may be based on surface plasmon resonance, particle plasmon resonance, a polarisation change or a change in the optical absorption coefficient.

A change in permittivity in the response region pattern 106, 108 is typically based on the bonding between the analyte and signature molecule. A change in permittivity can be detected as a change in an optical and/or electric property of the response region pattern 106, 108.

A change in an electric property can be a change in resistance or impedance, for instance, that can be measured with a prior-art external measuring device.

A change in an optical property can be a change in the refractive index that can be measured by utilising interferometrics, such as Young's interferometrics, or some other method measuring a change in an optical distance.

In a thermal change, the interaction between the analyte and signature molecule produces a measurable temperature change in the biosensor 100.

In a mechanical change, the interaction between the analyte and signature molecule produces a measurable mechanical change in the biosensor. In one embodiment, the mechanical change changes the specific frequency of an oscillator in the biosensor, which can be measured.

The substrate of the biosensor 100 can be paper, polymer, glass, metal, or ceramics, for instance. The substrate can be processed by plasma processing or with some other surface treatment method to improve the contact between the sol-gel medium and surface.

With reference to FIG. 2, the micro-channel 202 of the biosensor can be a groove formed in the substrate 200 and made by laser ablation, hot pressing or pressing, for instance. The width 204 of the micro-channel 202 can vary from dozens of micrometers to millimetres. The depth 206 of the micro-channel 202 can vary from dozens of micrometers to half a millimetre. The present solution is, however, not limited to these width and depth figures, but the width 204 and depth 206 can be determined according to the properties and column structure of the used material and its transport mechanism in the micro-channel 202.

The micro-channel 202 can also be made of microcellulose patterned by pressing.

In one embodiment, the micro-channel 202 comprises microcolumns and each micro-column forms a sub-channel in the micro-channel. Micro-columns provide a wide effective micro-channel that utilises a capillary formed by narrow micro-columns. The width of the micro-columns can be 10 to 500 μm and their depth 20 to 500 μm. One micro-channel 202 can comprise thousands of micro-columns.

In one embodiment, the transport mechanism of material in the micro-channel 202 is based on a capillary mechanism. The width 204 of the micro-channel is then typically 100 to 200 μm and the depth 20 to 100 μm.

In one embodiment, the transport mechanism of material in the micro-channel 202 is based on the use of a pump, such as an injection pump. The typical pumping rate is 0.001 to 10 ml/min.

In one embodiment, the transport mechanism of material in the micro-channel 202 is based on a pressure difference between the supply region 114 and sol-gel response region pattern 106, 108. The pressure difference can be provided with air pumping or surge pumping, for example.

In one embodiment, the transport mechanism of material in the micro-channel 202 is based on a pH difference between the supply region 114 and sol-gel response region pattern 106, 108.

In one embodiment, the transport mechanism of material in the micro-channel 202 is based on a voltage difference between the supply region 114 and sol-gel response region pattern 106, 108.

In one embodiment, the micro-channel 202 is arranged to mix the material. The mixing can for instance be based on a column structure, or connecting several micro-channels, or both.

In one embodiment, the micro-channel 202 is arranged to separate the material. The separation can for instance be based on the different diffusion rates of different-sized molecules of the material in the micro-channel 202, or to a separation according to the size of the materials in various micro-channel and column structures.

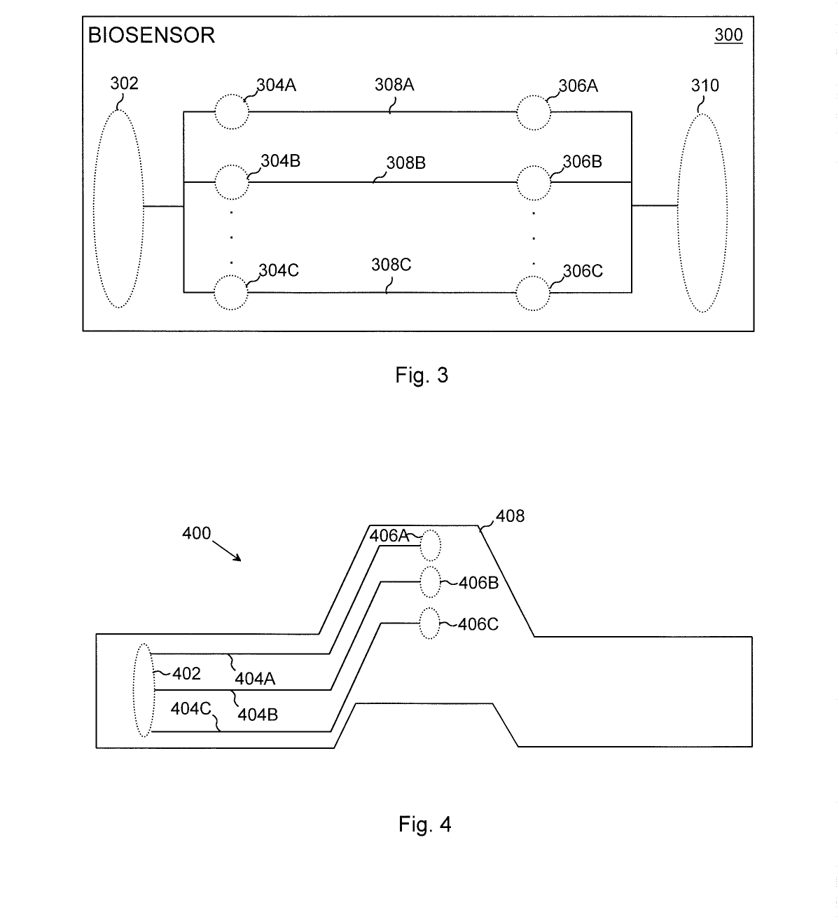

With reference to FIG. 3, the biosensor 300 can comprise several sol-gel response region patterns 306A to 306C that are connected through the micro-channel 308A to 308C to the supply region 302 and a secondary signature molecule region 304A to 304C. The supply region 302 receives biological material and supplies it to the micro-channel 308A to 308C. The secondary signature molecule region 304A to 304C contains a second signature molecule that, as the material flows, can go with the material, mix with it, and bond with the analyte in the material. The signature molecule in the sol-gel response region pattern 306A to 306C identifies and bonds the complex which is formed when the second signature molecule and the analyte bond. FIG. 3 also shows a collection region 310 to which any unbonded material is collected. Each sol-gel response region pattern 306A to 306C can be measured separately as shown in FIG. 1.

FIG. 4 shows an implementation of a biosensor 400 in which the biosensor 400 comprises a measuring adapter 408 for connecting the biosensor 400 to a measuring device 118. The measuring adapter 408 can be a protrusion in the biosensor 400, which is placed in the positioning structures of the measuring device 118. The sol-gel response region patterns 406A to 406C of the biosensor 400 then settle in the measuring system of the measuring device 118 in such a manner that they can be measured.

FIG. 4 also shows a supply region 402 and micro-channels 404A to 404C leading from the supply region to the sol-gel response region patterns 406A to 406C.

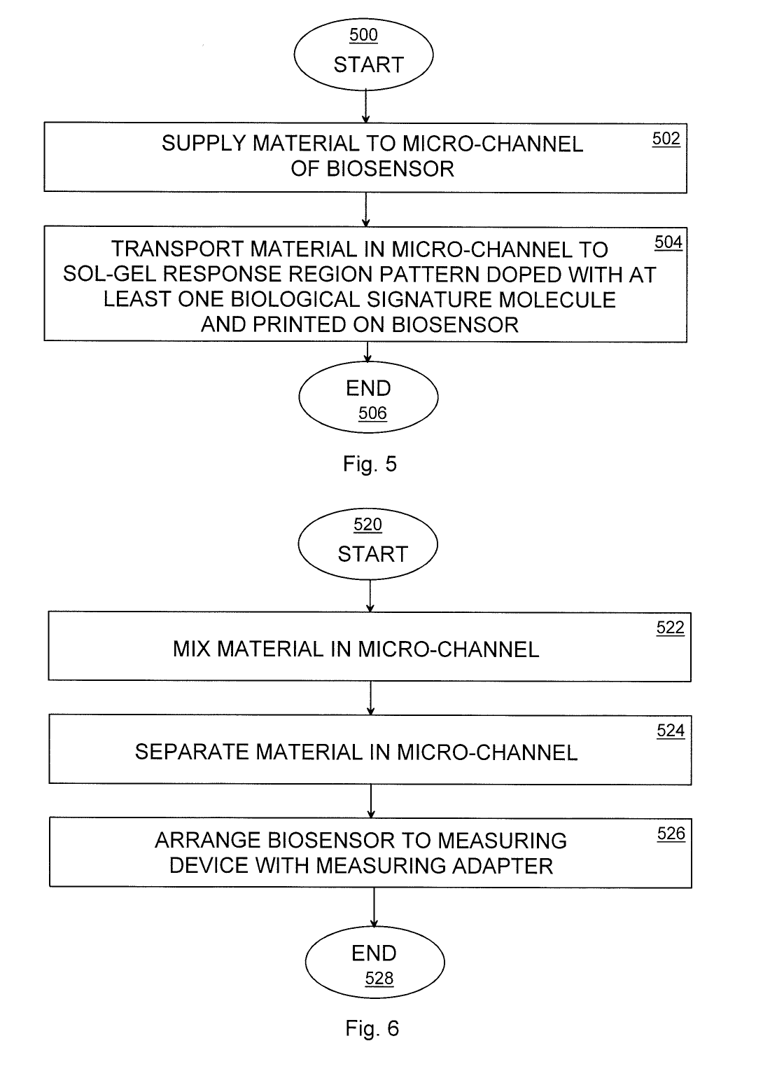

With reference to FIG. 5, let us examine embodiments of the method related to the operation of the biosensor.

The method starts in step 500.

In step 502, material is supplied to the micro-channel 104 of the biosensor 100.

In step 504, the material is transported in the micro-channel 104 to a sol-gel response region pattern 106, 108 doped with at least one biological signature molecule and printed on the biosensor.

The method ends in step 506.

With reference to FIG. 6, let us examine the embodiments of FIG. 5. The parts of the method in FIG. 6 can be freely set in the method of FIG. 5.

The method starts in step 520.

In step 522, material is mixed in the micro-channel 104.

In step 524, material is separated in the micro-channel 104.

In step 526, the biosensor is arranged to the measuring device 118 by means of a measuring adapter 408.

The method ends in step 528.

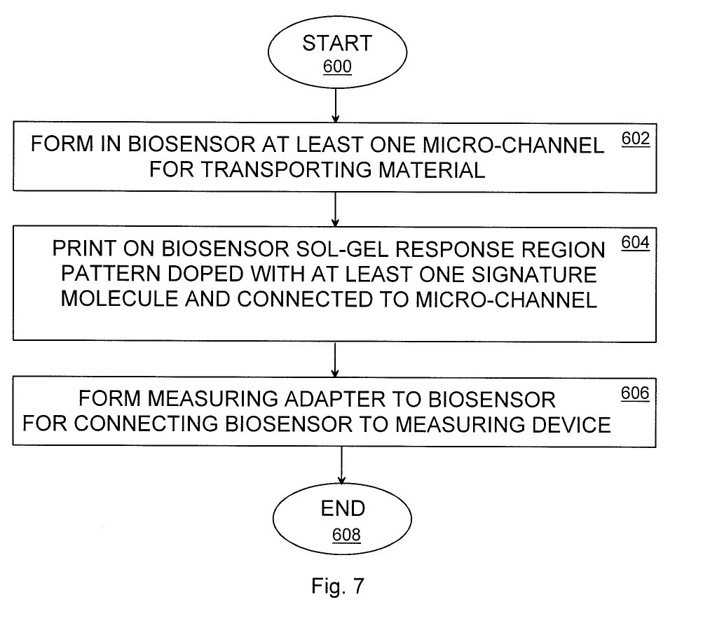

An embodiment of the manufacturing method of the biosensor is described with reference to FIG. 7.

The method starts in step 600.

In step 602, at least one micro-channel 104 for transporting material is formed in the biosensor 100.

In step 604, a sol-gel response region pattern 106, 108 doped with at least one biological signature molecule and connected to said at least one micro-channel 104 is printed on the biosensor 100.

In step 606, a measuring adapter 408 is formed on the biosensor 400 to connect the biosensor 400 to the measuring device 118.

The method ends in step 608.

Further, with reference to the methods of FIGS. 5, 6, and 7, in one embodiment the sol-gel response region pattern 106, 108 is printed on the biosensor 100 by using an ink transfer method.

In one embodiment, the biological signature molecule has a measurable response 110, 112 to at least one previously known component of the material.

In one embodiment, the signature molecule is selected in such a manner that the measurable response is at least one of the following: an optical radiation emission from the sol-gel response region pattern 106, 108, a change in the optical reflection coefficient in the sol-gel response region pattern 106, 108, a change in the permittivity in the sol-gel response region pattern 106, 108, a thermal change in the sol-gel response region pattern 106, 108, and a mechanical change in the sol-gel response region pattern 106, 108.

In one embodiment, the micro-channel 104, 202 is formed on the biosensor by grooving.

In one embodiment, the micro-channel 104, 202 is arranged to mix the material.

In one embodiment, the micro-channel 104, 202 is arranged to separate the material.

In one embodiment, the signature molecule is an antibody, antibody fragment, or antibody produced by gene technology processes (recombinant antibody).

In one embodiment, the signature molecule is a combination of two different antibodies, antibody fragments, or recombinant antibodies or recombinant antibody fragments bonding an analyte.

In one embodiment, the biological signature molecule is a desired mixture of two or more antibodies, antibody fragments, or recombinant antibodies or recombinant antibody fragments.

Even though the invention has above been described with reference to an example according to the attached drawings, it is clear that the invention is not restricted to it, but can be modified in many ways within the scope of the attached claims.

1. A biosensor for analysing material, the biosensor comprising:
at least one sol-gel response region pattern doped with a biological signature molecule and printed on the biosensor; and
at least one micro-channel for transporting the material to said at least one sol-gel response region pattern.

2. A biosensor as claimed in claim 1, wherein the sol-gel response region pattern is printed using an ink transfer method.

3. A biosensor as claimed in claim 1, wherein the biological signature molecule has a measurable response to at least one previously known component of the material.

4. A biosensor as claimed in claim 3, wherein the signature molecule is selected in such a manner that the measurable response is at least one of the following: an optical radiation emission from the sol-gel response region pattern, a change in the optical reflection coefficient in the sol-gel response region pattern, a change in the permittivity in the sol-gel response region pattern, a thermal change in the sol-gel response region pattern, and a mechanical change in the sol-gel response region pattern.

5. A biosensor as claimed in claim 1, wherein the micro-channel is a groove in the structure of the biosensor.

6. A biosensor as claimed in claim 1, c h a r a c t c r is c d in that wherein the micro-channel is arranged to mix the material.

7. A biosensor as claimed in claim 1, wherein the micro-channel is arranged to separate the material.

8. A biosensor as claimed in claim 1, wherein the biological signature molecule is an antibody or antibody fragment.

9. A biosensor as claimed in claim 1, wherein the biosensor comprises a measuring adapter for connecting the biosensor to a measuring device.

10. A method for analysing material, the method comprising:
supplying material to the micro-channel of the biosensor; and
transporting the material in the micro-channel to a sol-gel response region pattern doped with at least one biological signature molecule and printed on the biosensor.

11. A method as claimed in claim 10, the method further comprising the sol-gel response region pattern is printed using an ink transfer method.

12. A method as claimed in claim 10, the method further comprising the biological signature molecule has a measurable response to at least one previously known component of the material.

13. A method as claimed in claim 12, the method further comprising the signature molecule is selected in such a manner that the measurable response is at least one of the following: an optical radiation emission from the sol-gel response region pattern, a change in the optical reflection coefficient in the sol-gel response region pattern, a change in the permittivity in the sol-gel response region pattern, a thermal change in the sol-gel response region pattern, and a mechanical change in the sol-gel response region pattern.

14. A method as claimed in claim 10, the method further comprising the micro-channel is a groove in the structure of the biosensor.

15. A method as claimed in claim 10, the method further comprising mixing the material in the micro-channel.

16. A method as claimed in claim 10, the method further comprising separating the material in the micro-channel.

17. A method as claimed in claim 10, wherein the biological signature molecule is an antibody or antibody fragment.

18. A method as claimed in claim 10, wherein the biosensor is arranged to the measuring device with a measuring adapter.

19. A method for manufacturing a biosensor, the method comprising:
forming into the biosensor at least one micro-channel for transporting material; and
printing on the biosensor a sol-gel response region pattern doped with at least one biological signature molecule and connected to said at least one micro-channel.

20. A method as claimed in claim 19, the method further comprising printing the sol-gel response region pattern by using an ink transfer method.

21. A method as claimed in claim 19, wherein the biological signature molecule has a measurable response to at least one previously known component of the material.

22. A method as claimed in claim 21, wherein the signature molecule is selected in such a manner that the measurable response is at least one of the following: an optical radiation emission from the sol-gel response region pattern, a change in the optical reflection coefficient in the sol-gel response region pattern, a change in the permittivity in the sol-gel response region pattern, a thermal change in the sol-gel response region pattern, and a mechanical change in the sol-gel response region pattern.

23. A method as claimed in claim 19, method further comprising forming micro-channel in the biosensor by grooving.

24. A method as claimed in claim 19, the method further comprising mixing, in the micro-channel, material.

25. A method as claimed in claim 19, the method further comprising separating, in the micro-channel, the material.

26. A method as claimed in claim 19, wherein the biological signature molecule is an antibody or antibody fragment.

27. A method as claimed in claim 19, the method further comprising forming into the biosensor a measuring adapter for connecting the biosensor to a measuring device.
